(19) United States
(12) Patent Application Publication
(10) Pub. No.: US 2008/0234555 A1
(43) Pub. Date: Sep. 25, 2008

(54) PATIENT CARE SYSTEM

(75) Inventors: Patrick Lafleche, Kalamazoo, MI (US); Chad Rohrer, Chicago, IL (US)

Correspondence Address: VAN DYKE, GARDNER, LINN & BURKHART, LLP, SUITE 207, 2851 CHARLEVOIX DRIVE, S.E., GRAND RAPIDS, MI, 49546, US

(73) Assignee: STRYKER CORPORATION, Kalamazoo, MI (US)

(21) Appl. No.: 12/054,017

(22) Filed: Mar. 24, 2008

Related U.S. Application Data
(60) Provisional application No. 60/919,692, filed on Mar. 23, 2007.

(51) Int. Cl. A61B 5/00

(52) U.S. Cl. 600/300

(57) ABSTRACT

A patient care system includes a support, with a lower portion and an upper portion, and a controller. The upper portion is removably mounted to the lower portion wherein the upper portion may be removed from the lower portion. The lower portion of the support is adapted for being supported on a floor or ground surface. The upper portion is also adapted to mount to a fixture when removed from the lower portion and further includes an input connector for coupling to a monitoring device. The controller is in communication with the input connector, which is adapted for coupling the controller with a monitoring device for receiving data from the monitoring device for one of (1) storing, (2) monitoring, and (3) analyzing the received data.

The present application claims the benefit of provisional application, entitled PATIENT CONTROL APPARATUS, filed Mar. 23, 2007, Ser. No. 60/919,692, which is herein incorporated by reference in its entirety.

### TECHNICAL FIELD AND BACKGROUND OF THE INVENTION

The present invention relates to patient equipment and, more specifically, a to patient care system.

ICU rooms typically use several devices or pieces of equipment to monitor a patient and to deliver fluids to a patient. Many of these devices and equipment need to stay with the patient at all times. However, many of these devices require power so that when a patient is moved from one location to another location, the device or devices many need to be temporarily disconnected from the electrical outlet and, hence, the patient is temporarily no longer monitored and/or treated.

While IV poles facilitate the transport of IV bags and pumps along with a patient while the patient is transferred between locations, a care giver must push the patient's bed with one hand and pull the IV pole with the other hand, which can make maneuvering through a hospital difficult.

Accordingly there is a need for an improved way to handle patient care devices and equipment that will not only assure that the patient will have full access to the devices and equipment while being transported through the hospital, but also reduce the burden on caregivers when handling patients. Further, with this multitude of equipment, a large amount of information is generated, which must be monitored in order to provide proper care and treatment for a patient. Accordingly, there is a need to integrate such information, for example the information that is available in an ICU room, to help a caregiver better assess a patient's condition so that appropriate action can be taken as quickly as possible.

### SUMMARY OF THE INVENTION

The present invention provides a method and apparatus that will facilitate handling of a patient and the peripheral devices needed to care for the patient.

In one form of the invention, a patient care system includes a controller and a patient control apparatus with a housing, a user actuatable input device at the housing in communication with the controller, and a display, with the controller in communication with the display and operable to display an image at the display. Also provided is an input connector at the housing. The input connector is also in communication with the controller and allows one or more peripheral devices to communicate with or be controlled by the controller. The apparatus also includes a base and a pole extending upwardly from the base. The housing is adapted to be releasably mounted at the pole and also at a fixture, such as a bed or a boom, so that the housing may be supported by the pole or by the fixture when the housing is removed from the pole.

In a further aspect, the user actuatable input device comprises a key pad that has a plurality of input buttons, which is in communication with the controller. In this manner, a user may input commands or information into the controller.

In another aspect, the display comprises a touch screen. For example, the touch screen may include one or more touch sensitive areas that may form a plurality of user actuatable input devices and which send signals to the controller. For example, a portion of the touch screen may be used as a menu or menu selector. Another portion may function as a selector button.

Alternately, the display may display information gathered by the controller, for example information input in to the controller by a caregiver or information received by the controller from, for example, a peripheral device, such as a patient monitoring device.

In other aspects, the apparatus also includes a support structure, for example a hook, for supporting or suspending a peripheral device at the apparatus. Optionally, the apparatus includes a second support structure for supporting or suspending a peripheral device at the apparatus. It should be understood that multiple support structures may be provided. In addition, at least one of the support structures may be adjustable along one or more axes and/or in any plane. For example, the support structure may be vertically or horizontally adjustable or both, including horizontally adjustable about a vertical central axis of the apparatus or vertically adjustable in a direction generally parallel to the vertical central axis.

In a further aspect, the support structure may be coupled to the housing wherein when the housing is removed from the pole, the support structure is removed along with the housing. Further, the housing includes an upper portion and a lower portion. The upper portion may include the controller, the display, and the user actuatable device. The lower portion may form a mount for mounting the housing to the pole or to a fixture.

According to other aspects, the apparatus further may include an electrical outlet for electrically coupling a peripheral device to an energy source. For example, the energy source may comprise an external energy source such that the electrical outlet electrically couples the peripheral device to the external source.

Alternately or in addition, the apparatus may include an onboard energy source, such as a battery, including a rechargeable battery. For example, the controller may recharge the rechargeable battery when the electrical outlet is coupled to an external energy source, and when the electrical outlet is decoupled from the external energy source the controller supplies electrical current to the electrical outlet from the battery.

For example, the onboard energy source and electrical outlet may be located in the housing.

According to yet other aspects, the pole may be vertically adjustable. For example, the apparatus may include an actuator for extending the pole. Further, the apparatus may include a foot operated device for actuating the actuator. Alternately, or addition, the apparatus may include a spring assist device, such as a spring or gas cylinder, which reduces the amount of force needed to extend the pole.

In yet other aspects, the base may be configured to rest on a floor so that the base stationary or have bearings, such as casters, that make contact with the floor to facilitate movement of the apparatus. Further the bearings may be retractable and selectively deployed, for example, when an attendant or user wishes to move the apparatus, which may enhance the stability of the apparatus.

In another form of the invention, a patient care system includes a patient control with a pole and a base, with the pole extending upwardly from the base. The pole has an upper portion and a lower portion. The upper portion is removably mounted to the lower portion wherein the upper portion may be removed from the lower portion. The upper portion is adapted to mount to a fixture when removed from the lower portion and includes an electrically powered device or a power supply.

For example, the upper portion may include a controller, an electrical outlet, and an onboard power supply, with the outlet for coupling a peripheral device to the onboard power supply. Alternately, or in addition, the upper portion may include a printer, such as a thermal transfer printer for printing clinical documentation, for example.

In one aspect, the controller is adapted to couple to an external energy source. When the controller is coupled to the external energy source, the controller supplies electrical current to the electrical outlet from the external energy source. When the controller is decoupled from the external energy source, the controller supplies electrical current to the electrical outlet from the onboard energy source.

In a further aspect, the onboard energy source is located at the upper portion of the pole.

According to yet another aspect, the apparatus includes an input connector that is in communication with the controller and that allows one or more peripheral devices to be in communication with the controller. Optionally, the apparatus includes a plurality of input connectors. Further, the communication can be unidirectional or bidirectional. For example, the controller may receive data from the peripheral device, including bed data or patient data described more fully below, or may send data, including bed data or patient data, or send control signals to control the peripheral device.

According to a further aspect, a display is provided at the apparatus, which is in communication with the controller, which may be configured to display various types of information, such as status information, and/or display messages. For example, the display may be provided at the upper portion of the pole. In addition, the display may be a touch screen display, which has one or more touch sensitive areas and is communication with the controller to allow a user to communicate with the controller through the touch screen.

In further aspects, the patient control apparatus further includes a plurality of user actuatable devices, such as buttons, switches, or keys on a keypad, which are in communication with the controller and allow a user to communicate with the controller. Alternately or in addition, the apparatus my incorporate voice activation software, for example in the onboard controller or in another controller, which allows a user to control or input data into the controller through voice commands.

In any of these combinations, the pole may be vertically adjustable. For example, the lower portion of the pole may be vertically adjustable and further may include an actuator to adjust the height of the lower portion relative to the base.

In any of the above combinations, the controller may receive data from an input source, such as a peripheral device. The data may include bed data or patient data, for example, patient data collected or being collected by a patient monitoring device, for example, a blood pressure monitoring device, an oxygen detector, such as a pulse oximeter, to name a few. The controller may include a memory device and store the received patient data in the memory device. Further, if more than one peripheral devices is in communication with the controller, the controller may be configured to organize the data by the type of condition being monitored, by the peripheral device, or by other criteria. For example, the data may include a patient's name, identification, location, admittance date, or the like. Further, the data may include the type of data being monitored and the patient's status of the type of conditions being monitored.

The stored patient data may then be formatted by the controller. For example, the controller may create a report or a display, such as spreadsheet or graphical layout, or several displays or windows, with the stored patient data, which can be printed or transmitted or made available to others, for example, through a cable or by wireless transmission, including radio frequency or infrared transmission or inductive coupling. For example, a nurse or doctor at another location in the hospital or a family member may access the data and, therefore, monitor or view the data through a data link to the controller, with the data link as noted being either a cable or wireless link, or the like. In addition, the controller may be configured to allow a caregiver to select, deselect, reorganize the data or arrange the data in a format the she or he chooses. In this manner, a caregiver may have input into the type or report or output generated by the controller. Further, the caregiver may input data into the controller using the user interface device to populate, for example a report.

In addition or alternatively, the data in its raw or unformatted form may be printed, transmitted or accessed.

In any of the above combinations, the apparatus may include or be coupled to a printer.

As noted the controller may generate control signals to control a peripheral device and/or may generate an alarm. For example, the controller may have stored in the memory device reference data, for example in a look-up table or database, associated with the condition being detected and/or monitored by the peripheral device, and against which the controller may compare the patient data. The reference data may include values or ranges against which the controller may compare the received and/or stored data, for example patient data. If the controller determines that the patient data deviates from the reference data—for example exceeds a maximum reference data value, is below a minimum reference data value, or falls out of a range of reference data values—then the controller may actuate an alarm or some sort of notification, so that an attendant will be alerted to the unacceptable patient data. Alarms may include audible or visual alarms, including messages displayed on the display.

Alternately, the controller may have intelligence, for example software, that uses equations or other logic, to evaluate the incoming data. This may include software that monitors a patient's trends, including subtle trends and drastic changes over time. For example, in the ICU it is important to get a handle on the hemodynamic vitals of patient and be able to look at a patient's trends to predict when the patient can be moved to another area of the hospital, typically the Med/Surge area.

Further, the controller may have stored in the memory device suggested treatments or actions, for example a look-up table or database, for the type of condition being monitored and for the deviation that is determined by the controller. The controller may then determine based on the deviation and the condition being monitored what is a suggested treatment or action and, further, instruct a caregiver of the suggested treatment or action by way of the display or generate an audible instruction using prerecorded instructions stored on the controller (or its associate memory device or a recording device, such as an MP3 player or the like) and through a microphone, also provided at the patient control. Alternately, the controller can generate signals associated with the suggested treatment to actuate a peripheral device to implement the suggested treatment or action.

Also, in any of the apparatus described above, the controller may include a data or signal link, such as a cable, a transmitter or transceiver, or wiring, to allow the controller to communicate with other equipment in the area of a bed, for example, in a hospital room, to manage them from a tracking standpoint.

Accordingly, the present invention provides a system and apparatus for supporting and powering various electrically powered patient care devices and equipment. Further, the system enables communication between the various devices and equipment supported by the apparatus and devices, such as sensors, provided at or on a bed, and further between the controller and a caregiver, including a caregiver remote from the patient, as an alert system or monitoring system. By incorporating an onboard energy source, the apparatus allows all equipment devices to work in transport. Further, the present invention provides a user with a controller that will be able to manage interaction between equipment, as well as manage information related to the patient and/or the bed.

These and other objects, advantages, purposes, and features of the invention will become more apparent from the study of the following description taken in conjunction with the drawings.

### DESCRIPTION OF THE DRAWINGS

FIG. 1 is an elevation view of a first embodiment of the patient care system and apparatus of the present invention;

FIG. 2 is an elevation view of another embodiment of the patient care system and apparatus of the present invention;

FIG. 3 is a perspective view of the apparatus with a plurality of devices, such as a pump and a blood pressure monitor mounted to the apparatus and, further, with respect to devices plugged into the connectors provided at the apparatus;

FIG. 4 illustrates a portion of the apparatus mounted to a patient support;

FIG. 5 illustrates another embodiment of the patient care system and apparatus of the present invention incorporating a handle into the apparatus to facilitate handling; and

FIG. 6 is a schematic flowchart of a data monitoring function of the patient care system and apparatus and the optional control functions of the controller.

### DETAILED DESCRIPTION OF THE PREFERRED EMBODIMENTS

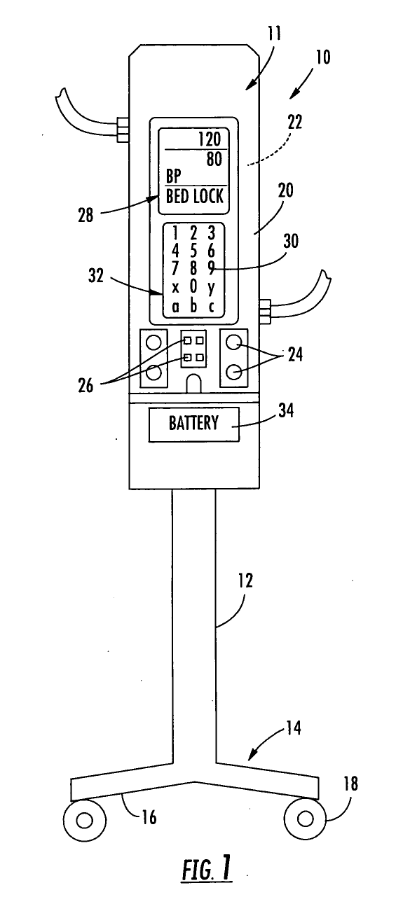

Referring to FIG. 1, the numeral 10 generally designates a patient care system of the present invention. As will be more fully described below, patient care system 10 includes a patent support apparatus 11 that provides a central hub for mounting a plurality of patient monitoring devices and/or fluid delivery devices, such as an IV bag and IV pump, adjacent a patient support, such as a bed and, further, which allows at least a portion of the apparatus to then be mounted on the patient support so that the patient monitoring devices and fluid delivery devices can be transported along with the patient on the patient support. In addition, patient care system 10 optionally directs electrical current to various electrically powered devices mounted or suspended at apparatus 11 either from an external power source, such as a conventional AC power source, or an onboard power supply, such as a battery. Further, patient care system 10 is adapted to provide a communication hub between the patient monitoring devices suspended or mounted at the apparatus and devices, such as sensors, either mounted at the bed or on the patient, and also between monitoring devices at, for example, a patient's bed, surgical table, stretcher, or cot or the like.

As a communication hub, patient care system 10 may collect, monitor, analyze and respond to patient data to either alert a caregiver of a patient's condition, notify a caregiver of a suggested action, such as a suggested treatment, actuate a peripheral device to perform a suggested action, and allow access to a caregiver or family member to the data being collected, monitored or analyzed by the controller, either in an unformatted form or in a formatted form, for example in the form of a report. While reference hereinafter will be made to the use of the patient care system in the context of a bed, it should be understood that the system of the present invention may be used in conjunction with other patient supports, including stretchers, wheelchairs, cots, and surgical tables or the like.

As best seen in FIG. 1, apparatus 11 includes a pole 12 and a base 14. Base 14 includes a plurality of radially extending arms 16, each with a caster 18 so that apparatus 11 can be easily moved across a floor, such as a hospital floor. It should be understood that the base may be adapted to rest on the ground directly or be fixed to the floor or may include retractable bearings so that when a caregiver or patient wishes to move the apparatus, the bearings are then deployed to engage the floor. Base 14 includes a plurality of arms, with each arm downwardly angled from pole 12 to provide a compact footprint having a total lateral span that is less than a conventional IV pole base. In this manner, base 14 provides a narrow base that facilitates maneuvering of apparatus.

Pole 12 includes a housing 20 that provides a power and communication hub for each of the devices mounted or suspended at apparatus 11. Housing 20 may be formed as a separate component that may then be mounted to the pole or it may be integrally formed with the pole, for example, from an upper portion of the pole as will be more fully described below.

System 10 also includes a controller 22. Controller 22 may be mounted in housing 20 and further may be removably mounted in housing 20 so that it may be removed and mounted, for example, at the patient support or at another fixture. Further when removed, the controller 22 may still be linked, either mechanically through a cable or wiring or wirelessly linked to the devices in and at housing 20 to provide communication between the controller and various devices in or at housing 20. Alternately, the controller may comprise a separate controller from apparatus 11, for example, a controller already present on a bed or the like, and simply being in communication with devices at the apparatus.

Controller 22 includes a microprocessor, software, a memory storage device, and circuitry for powering or communicating with one or more devices. When mounted in housing 20, those devices may include electrical outlets 24 so that devices that are plugged into the outlets may be powered by controller 22. Further, controller 22 is in communication with a plurality of connectors 26, also provided at housing 20, which allow two-way communication between one or more devices, which is plugged into a respective connector, and the controller. In the illustrated embodiment, connectors 26 comprise RJ 45 connectors. However, it should be understood that other suitable connectors may be used.

In addition, mounted at housing 20 is a display 28 which is in communication with controller 22 and displays images, such as alphanumeric text, spreadsheets, windows, video streams, or icons, or the like relating to the patient's status or treatment protocol, as well as patient trend data, as will be more fully described below. Further, as will be more fully described, display 28 may display messages or instructions to a caregiver. Display 28 optionally comprises a display screen, including for example a touch screen display, which may include a plurality of touch sensitive areas or regions on the screen that form user actuatable input devices, which allow a user to input data, including patient data to populate the controller with data, for example data from peripheral devices not coupled to the controller, select treatments, or otherwise communicate with controller 22. For example, a portion of the touch screen may be used as a menu. Another portion may provide a selector function.

Further optionally provided at housing 20 independent of the touch screen are one or more other user actuatable input devices 30, which in the illustrated embodiment comprise keys of a key pad 32. Other user actuatable devices may include buttons or switches. The keys may include alphanumeric keys, a menu key, a selector key, or the like. User actuatable input devices 30 similarly allow a user to input data, enter commands, select treatments, or otherwise communicate with controller 22. Alternately, or in addition, apparatus 10 may incorporate voice activation software, for example in controller 22 or in another controller in communication with controller 22, which allows a user to control or input data into the controller through voice commands.

As noted, display 28, user actuatable input devices 30, and the touch screen are in communication with controller 22, which displays one or more images at display 28. User actuatable input devices 30 and/or the touch screen allow a user to input data into the controller or otherwise communicate with the controller or other devices which are in communication with controller 22. For example, a user may input a patient's name, age, weight, or select or actuate a function, such as the initiation of a blood pressure reading, an EKG reading, a temperature reading, or the like. Further, as will be more fully described below, a user may organize or select or deselect data for inclusion in a report format, with the report format dictated by the controller or by the use.

As noted above, controller 22 is in communication with connectors 26 to allow communication with devices mounted or suspended at apparatus 11. In addition, controller 22 is in communication with one or more devices provided at a bed through either wireless communication system, such as LAN (local area network) or PAN (personal area network, such as Bluetooth), or hardwired for example through a cable, such as an Ethernet cable, that is plugged into one of the connectors 26 on one end and plugged into a similar port or connector at the bed, which is in communication with the bed communication network. For example, controller 22 may be in communication through the bed network with various sensing devices, including bed sensing devices or patient sensing devices, provided at the bed. For example, the bed sensing devices may include pressure sensors, angle sensors, moisture sensors, temperature sensors, motion sensors, or the like, which sense the status of a bed component or condition of the bed. The patient sensing devices may include temperature sensors, oxygen sensors or monitors, such as a pulse oximeter, EKG sensors or monitors, pressure sensors or monitors, including pressure cuffs, or the like, or a glucose monitor, which monitor a condition of a patient, such as the patient's temperature, oxygen levels, blood pressure, glucose levels, weight, movement, heart rate, electrical signals in the brain, or the like.

In some situations, such as a pressure cuff, the cuff may be directly coupled to the controller through a connector 26. In this manner, controller 22 may use this information obtained from any one of the sensing devices to control or provide input to one of the peripheral devices that is in communication with the controller. For example, when a blood pressure reading is initiated, the controller 22 may check the height of the bed and/or the angle of the Fowler section of the bed and adjust a blood pressure monitor, which is connected to one of the connectors and hence in communication with controller 22, to take into account the position of the patient to assure a more accurate blood pressure reading.

Display 28 may also be used to display the status of each equipment or device coupled to the controller and, further, help manage interaction between the devices and the controller. Additionally, display 28 may allow a user to see patient information and as noted enter data or information about the patient. Optionally, as noted, keypad 32 may include a menu button so that multiple functions can be displayed and selected by a selector button or by a touch sensitive area of display 28 in the case of a touch screen display.

As noted above, controller 22 electrically powers the various devices plugged into the electrical outlets 24 provided at housing 20 and powers the outlets either from an external power source, as noted above, or an onboard power source. In preferred form, housing 20 includes an onboard energy source, such as a battery, including a rechargeable battery, which will allow the various devices that are plugged into the controller or mounted to or suspended at the apparatus (11) to be powered when a conventional wall power supply is not available.

In one form, housing 20 is preferably removably mounted to pole 12 so that housing 20 may be mounted to a bed, as will be more fully described in reference to the second embodiment. In this manner, when a patient needs to be transported from one area or location in the hospital to another area or location in the hospital, the controller along with the energy source and the various devices that are plugged into the controller may be moved with a patient along with the bed, while still being powered.

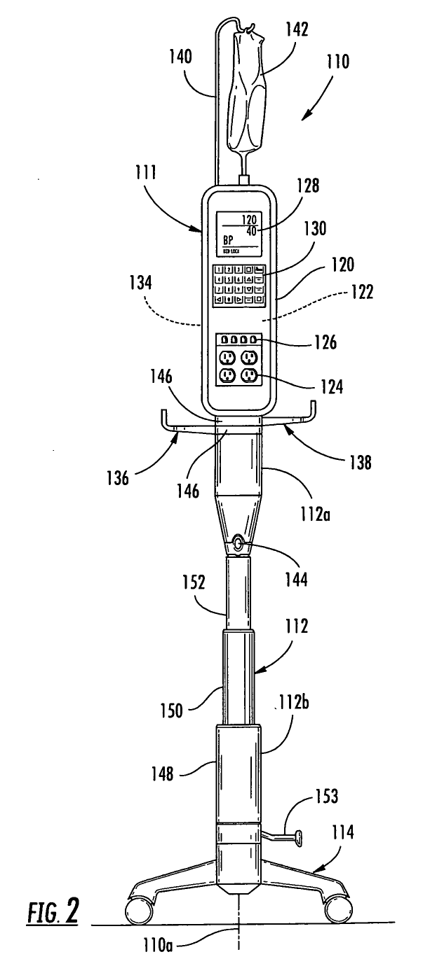

Referring to FIG. 2, the numeral 110 generally designates another embodiment of the patient care system of the present invention. Patient care system 110 is similar to patient care system 10 and includes a controller 122 and a patient care apparatus 111 that provides a centralized hub for mounting a plurality of patient monitoring devices and/or fluid delivery devices adjacent a bed and, further, which allows at least a portion of the apparatus to be then mounted to the bed B so that the patient monitoring devices and fluid delivery device(s) can be transported along with the patient on the bed. For any features or functions not specifically described in reference to the second embodiment reference is made to the first embodiment.

Patient apparatus 111 includes a pole 112 and a base 114. Pole 112 includes an upper portion 112a and a lower portion 112b, which is secured to base 114. Base 114 is also compact and may incorporate bearings or be stationary and rest directly on the ground, similar to base 14. Upper portion 112a of pole 112 is removably mounted to lower portion 112b and includes or forms a housing 120.

Similar to the previous embodiment, housing 120 may house controller 122, which delivers power to a plurality of electrical outlets 124 and is communication with a plurality of connectors 126, such as RJ 45 connectors, provided at housing 120. Further, housing 120 includes a display 128, such as a touch screen display, and a plurality of user actuatable input devices 130, which allow a user to communicate with the controller and, for example, input data relative to the patient into the controller or select or actuate a function to be performed on the patient, such as a blood pressure reading or EKG reading or the like, as described above. This data may or may not be displayed at display 128.

In addition, housing 120 optionally includes an onboard power supply 134 that supplies power to the respective electrical outlets 124 provided at housing 120 when an external power source is unavailable or not accessed. Controller 122 is also adapted to couple to an external energy source, similar to system 10, so that controller 122 optionally directs electrical current to the peripheral devices plugged into outlets 124 from the external energy source when it is available (and accessed) or from the onboard energy source when the external energy source is unavailable or not accessed.

In addition, upper portion 112a may include a plurality of support structures, such as hooks 136 and 138, which allow devices to be releasably mounted or suspended at apparatus 111. An additional support, such as hook 140 may also be provided, which extends above housing 120, which is configured to hold, for example an IV bag 142 for delivering fluid to a patient, with its optional corresponding pump suspended at one of the hooks 136 or 138. The support structures may be fixed or adjustable, including adjustable along any axis or in any plane to thereby accommodate a wide variety of different devices or objects to be supported at apparatus 111

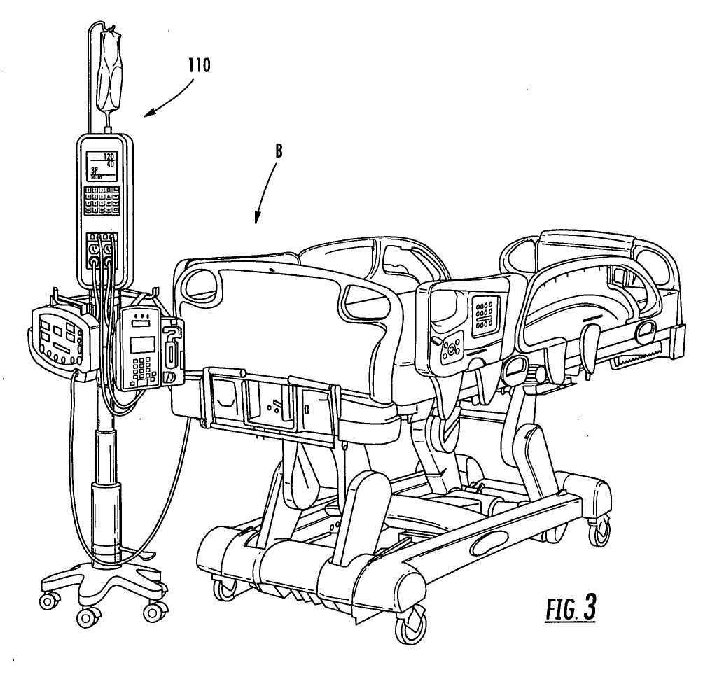

As best seen in FIG. 3, upper portion 112a may be disconnected from lower portion 112b of pole 112 so that upper portion 112a may be mounted to bed B on, for example, a post or other support structure. The post or support structure may be provided on the bed, such as on the bed frame, including for example at the head end of the bed frame or on another fixture in the room, such as a boom or the like. Further, it should be understood that the post or other support structure may be provided anywhere about the bed, including on a side rail, a footboard, a headboard, or on the base of the bed. In the illustrated embodiment, the lower end of upper portion 112a comprises a tubular member, such as a tapered cylindrical member, with an opening or receptacle at its lowermost end that forms a mount and receives the upper end of lower portion 112b. However, it should be understood that a separate mount may be provided.

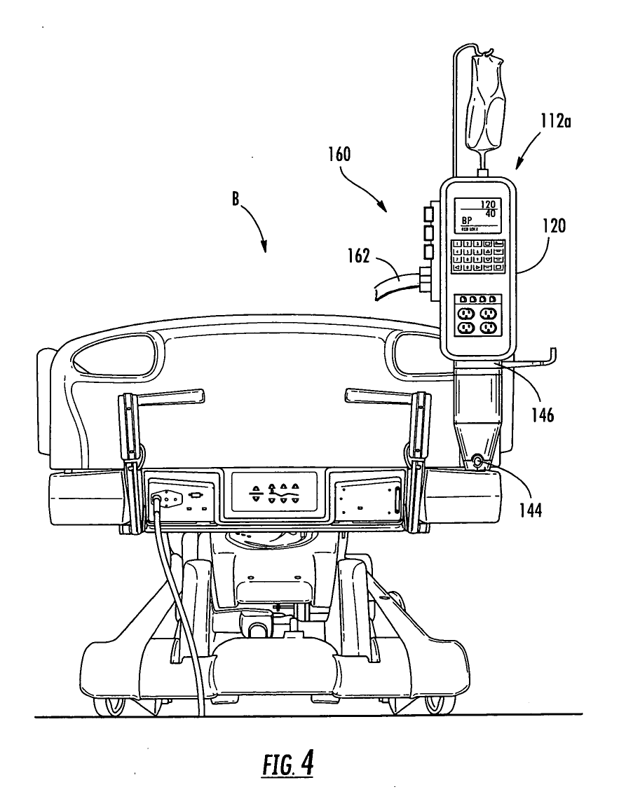

Optionally, upper portion 112a includes a latch mechanism 144 (FIG. 4), such as a spring biased pin, to securely fasten upper portion 112a to lower portion 112b when upper portion 112a is mounted over lower portion 112b of post 112 and, further, to releasably engage the support structure provided on the bed.

In the illustrated embodiment, each hook 136 and 138 is mounted to upper portion 112a of post 112 so that when upper portion 112a is removed from lower portion 112b the hooks will be transported along with housing. Referring to FIGS. 2 and 3, each hook 136 and 138 is mounted to portion 112a by a collar 146, which is either fixedly mounted to upper portion 112a or, as noted above, may be rotationally mounted about upper portion 112a to allow the respective hook to be pivoted about axis 110a. Hooks 136 and 138 may also be mounted to provide vertical adjustment of the respective hooks relative to the longitudinal axis 110a. Similarly, hook 140 may be mounted in a sleeve provided in housing 120. For example, a suitable sleeve may comprise a frictional sleeve with a threaded collar that frictionally clamps the hook in position when tightened but when loosened allows the hook to be vertically adjusted to accommodate different size bags, as would be understood by those skilled in the art.

In the illustrated embodiment, lower portion 112b of post 112 is formed from nested tubular members 148, 150, and 152, which may be telescoping to provide vertical adjustment of upper portion 112a relative to base 114. For example, tubular members 150 and 152 may be extended relative to sleeve 148 by an actuator, such as screw drive device, a chain, a cylinder, including a pneumatic cylinder or hydraulic cylinder, or by a servomotor. Therefore, adjustment of the pole height may be achieved through manual, electric, pneumatic, or hydraulic actuation. Further, lower portion 112b may include a foot pedal 153, which when moved to a first position actuates the actuator to extend tubular members 150 and 152 with respect to member 148 and when moved to a second position retracts one or more members 152 and/or 150 into member 148. Controls to adjust the pole height may also be provided at housing 120 or on the side of lower portion 112b. For example, a user actuatable device, such as a button or switch or a portion of the touch screen, may be mounted at housing 120, which actuates the actuator. Also, set positions may be provided. For example, the controller may be in communication with the actuator and the user actuatable device, which includes set height positions, so that when the button or switch is depressed, the IV pole will extend to a pre-selected height. A second position of the button or the switch may return the IV pole to a lower pre-selected height. Alternately, two buttons may be provided-one associated with the first pre-selected height, and the second associated with the second pre-selected height. Similarly, a user actuatable device such as a button or switch may be mounted at lower portion 112b that actuates, either directly, or through the controller, the actuator. The apparatus may also, or instead, include a spring assist device, such as a mechanical spring or gas cylinder, which reduces the amount of force needed to extend the pole. Alternately, the pole may have a fixed height with no adjustment option as well.

Optionally, another foot pedal may be provided to provide a release mechanism for the latch mechanism that secures upper portion 112 to lower portion 112b so that the housing 120 may be disengaged from lower portion 112b of pole 112 when the pedal is depressed, for example.

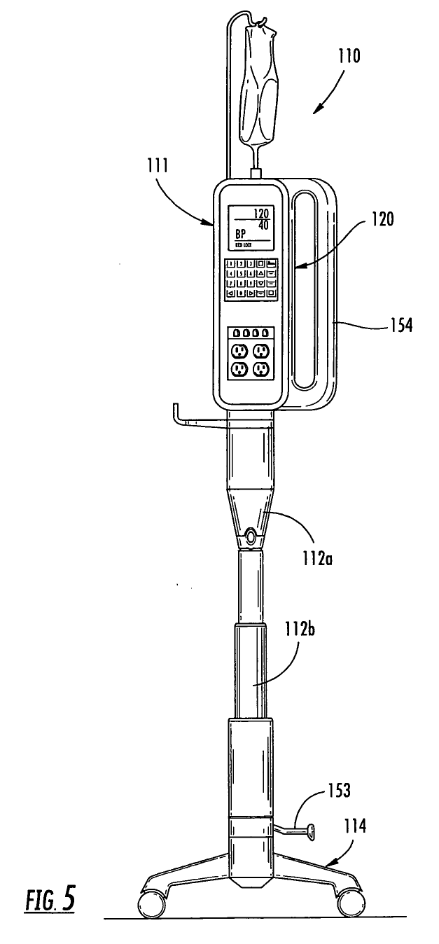

Referring to FIG. 5, patient care apparatus 111 may also incorporate a handle 154, for example, at housing 120, which facilitates the removal of housing 120 from lower portion 112b of pole 112. For example, handle 154 may be integrally formed with housing 120.

Further, as best seen in FIG. 4, a printer 160 that dispenses strips 162 of paper may be incorporated into apparatus 111 (or 11). Printer 160, for example, may be used to print clinical documentation or the like. Suitable printers may include thermal transfer printers, for example of the type sold by Panduit. Referring again to FIG. 4, printer 160 may be incorporated at housing 120. In the illustrated embodiment, printer 160 is partially enclosed in housing 120; however, printer 160 may be surface mounted or fully recessed in housing either so that its outer facing is generally flush or sub-flush with the outer surface of housing. Similar to the controller, printer 160 may be powered through an external power supply when one is available or, if not, through the on-board power supply. Further, while a printer with its own user actuatable input devices, such as buttons or a touch screen, and its own controller may be provided, the printer may comprise a printing mechanism controlled through controller 122 and by the user actuatable input devices mounted or provided at housing, such as the touch screen 128 or user actuatable input devices 130.

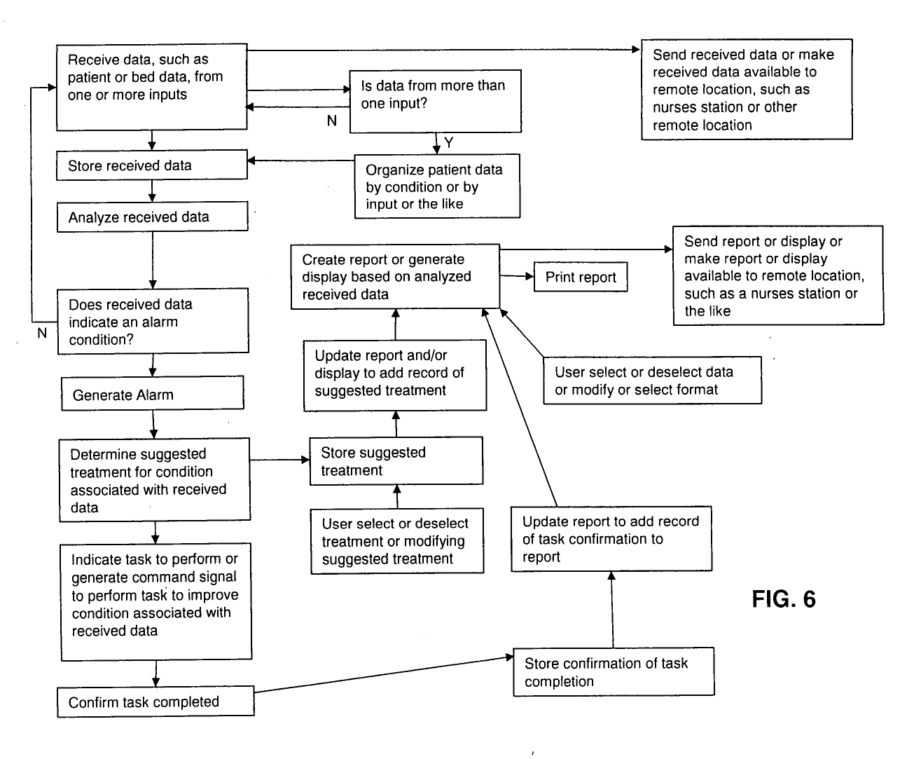

Referring to FIG. 6, in either embodiment the controller may receive data from an input, such as a peripheral device, including a bed monitoring device or a patient monitoring device, or a user using the user interface devices). The data may include bed data, for example, sensor readings, such as load sensor readings, that indicate, for example, a bed exit status by monitoring patient movement on the bed, moisture sensors for reading bed wetness, side rail position sensors, angle sensors, or the like. The data may also include patient data, for example, blood pressure data from a blood pressure monitoring device, oxygen levels from an oxygen detector, such as a pulse oximeter, to name a few. Further, the controller may be in communication with the hospital network of information, for example the electronic medical records (EMR) system, the pharmacy folders, or the laboratory files, or the like, to download and store further data in the controller

The stored data may then be formatted by the controller. The controller, for example, may create a report or display with the stored data, which can then be printed or transmitted or made available to others, for example, through a cable or by wireless transmission, including radio frequency or infrared transmission or inductive coupling. For example, a nurse or doctor at another location in the hospital (or a family member) may access the data and, therefore, monitor or view the data through a data link to the controller, with the data link, as noted, being either a cable or wireless link, or the like. In addition, the controller may display the information on display 28 or 128. For example, the display may include a spreadsheet or multiple spreadsheets, for example in a windows format, a graphical layout, including icons, charts, graphs, or gauges that are, for example, representative of the data collected by the controller or images collected by the controller, for example video streaming images or still images, which may be collected by a camera or video device mounted at the apparatus or elsewhere in the room.

In addition, if more data from more than one source than is transmitted or communicated to the controller, the controller may be configured to organize the data by the type of parameter being monitored or by the source of the data or other criteria. In addition or alternatively, the data in its unformatted or raw form may be printed, transmitted or accessed.

For example, if more than one input is in communication with the controller, the controller may be configured to organize the data by the type of condition being monitored, by the input source, or by other criteria, as noted above. The data may include a patient's name, identification, location, admittance date, as well as other previously stored data, such as lab results, pharmalogical data, or the like. Further, the data may include a reference to the type of data being monitored and the patient's status for the condition being monitored.

The stored patient data may then be formatted by the controller or formatted by user. For example, the controller may create a report or a display, such as spreadsheet or graphical layout, or several displays or windows, with the stored patient data, which can be printed or transmitted or made available to others, for example, through a cable or by wireless transmission, including radio frequency or infrared transmission or inductive coupling. For example, a nurse or doctor at another location in the hospital or a family member may access the data and, therefore, monitor or view the data through a data link to the controller, with the data link as noted being either a cable or wireless link, or the like. In addition, the controller may be configured to allow a caregiver to select, deselect, reorganize the data or arrange the data in a format that she or he chooses. In this manner, a caregiver may have input into the type of report or output generated by the controller. Further, the caregiver may input data into the controller using the user interface device to populate, for example a report. For example, when data is collected by a monitoring device that is not in communication with the controller, the caregiver or attendant may then input the data in to the controller.

As noted the controller may generate control signals to control a peripheral device and/or may generate an alarm. For example, the controller may have stored in its memory device reference data, such as patient reference data, for example in a look-up table or database or the like. The reference data may be organized based on the parameter being detected and/or monitored by the peripheral device. The reference data may include minimum and/or maximum values or ranges against which the controller may compare the patient data to determine the patient's condition or status relative to the parameter being monitored. If the controller determines that the patient data deviates from the reference data—for example exceeds a maximum reference data value or range of values or is below a minimum reference data value or range values—then the controller may actuate an alarm or some sort of notification, so that an attendant will be alerted to the unacceptable patient data. Alarms may include audible or visual alarms, including messages displayed on the display. Further, the alarm may be transmitted, either through a cable or a wireless transmission to another location, such as a nurses' station.

Alternately, the controller may have intelligence, for example in its software, that uses equations or other logic, to evaluate the incoming data. This may include software that monitors a patient's trends, including subtle trends and drastic changes over time. For example, in the ICU it is important to get a handle on the hemodynamic vitals of patient and be able to look at a patient's trends to predict when the patient can be moved to another area of the hospital, typically the Med/Surge area.

Further, the controller may have stored in the memory device suggested treatments or actions, for example in a look-up table or database, for the type of condition being monitored and for the deviation from the standard or reference data stored in the controller, which is determined by the controller. Therefore, based on the deviation and the condition being monitored, the controller may determine what is a suggested treatment or action. Further, the controller may instruct a caregiver of the suggested treatment or action by way of the display or by generating an audible instruction using prerecorded instructions stored on the controller (or its associate memory device or a recording device, such as an MP3 player or the like) and through a microphone, also provided at the apparatus (11 or 111). For example, the look-up table or database may have one or more suggested treatments or actions for a “high status”—that is when the controller determines that the patient data exceeds a maximum reference data value or range; or one or more suggested treatments or actions for a “low status”—that is when the controller detects that the patient data is below a minimum reference data value or range.

Alternately, the controller may have an equation or other logic or intelligence stored therein to determine what is a suggested action or treatment. Once a suggested action or treatment is determined, the suggested action or treatment may be stored in the memory device of the controller and recorded, for example in the report or spreadsheet, or the like. Further, once a suggested treatment is indicated, the user may confirm that the action has been taken where the action to be taken was by the user, which confirmation can also be stored and recorded in the report or spreadsheet or the like to create a record of the treatment being applied.

Alternately, the controller can generate signals associated with the suggested treatment to actuate a peripheral device to implement the suggested treatment or action. Therefore, where the controller initiates the action or treatment, the controller may confirm that the action was taken and store and record the confirmation as described above.

In addition to deselecting or selecting data or modifying the report format or spreadsheet format, the user may also select a particular treatment, deselect (or override) or modify the suggested treatment.

To provide communication with other peripheral devices or other equipment in the area of a bed, for example, in a hospital room, the controller may include a data or signal link, such as a cable, a transmitter or transceiver, or wiring, to allow the controller to communicate to and further manage them from a treatment implantation or tracking standpoint.

It should be understood that the various functions provided by the controller of the present invention are provide by software that is, for example, stored in the memory device of the controller and which operates in the microprocessor, though it should be understood depending on the processor, the software may also be resident in the microprocessor.

Accordingly, the present invention provides a system with a portable apparatus that is configured to attach at least a portion of the apparatus to a bed for transport with the bed and further incorporates an onboard energy source to ensure devices and equipment that are coupled to the apparatus will be powered during transport. Also, the system incorporates a controller that will automatically manage interaction in between equipment provided at the apparatus and the patient on the bed to ensure best treatment to patient is available. Further, patient data may be stored in the controller to help to define better treatment for the patient or define trends about the patient's healing process.

While several forms of the invention have been shown and described, other forms and features will now be apparent to those skilled in the art. For example, the various components of the apparatus, such as the pole and the housing, and the base, may be formed from plastic and/or metal components, or a combination of materials. Further, while the patent control apparatus of the present invention incorporates several electrically powered devices, such as the controller and the printer, other devices may also be incorporated. Alternately, the apparatus may include a single electrically powered device, for example, just the printer or just the controller. Therefore, it will be understood that the embodiments shown in the drawings and described above are merely for illustrative purposes, and are not intended to limit the scope of the invention which is defined by the claims which follow as interpreted under the principles of patent law including the doctrine of equivalents.

We claim:

1. A patient care system comprising:
a controller; and
an apparatus comprising:
a housing, said housing including a user actuatable input device in communication with said controller and a display, said controller operable to display an image at said display;
an input connector at said housing, said input connector in communication with said controller, and said input connector allowing one or peripheral devices to communicate with or be controlled by said controller;
a base; and
a support extending upwardly from said base, and said housing being removably supported by said support wherein said housing may be removed from said base and supported by a fixture.

2. The patient care system according to claim 1, wherein said input connector comprises a plurality of user input connectors, and said input connectors allowing a plurality of peripheral devices to communicate with or be controlled by said controller.

3. The patient care system according to claim 1, further comprising a key pad, said key pad having a plurality of input keys forming a plurality of user actuatable devices.

4. The patient care system according to claim 1, wherein said display comprises a touch screen, said touch screen including said user actuatable input device.

5. The patient care system according to claim 1, further comprising a support structure for supporting or suspending the peripheral device at said apparatus.

6. The patient care system according to claim 5, wherein said housing and said support comprise a pole, said pole having an upper pole portion and a lower pole portion, said housing being formed from said upper pole portion, and said support being formed from said lower pole portion.

7. The patient care system according to claim 6, wherein said housing includes an upper housing portion and a lower housing portion, said upper housing portion including said controller, said display, and said user actuatable device, said lower housing portion forming a mount for mounting said housing to said lower pole portion and for mounting said housing to a fixture when said housing is removed from said lower pole portion.

8. The patient care system according to claim 7, wherein said support structure is provided at said lower housing portion.

9. The patient care system according to claim 1, further comprising an electrical outlet at said apparatus electrically coupling to said controller.

10. The patient care system according to claim 9, further comprising an energy source, said controller electrically coupled to said energy source, and said electrical outlet for electrically coupling the peripheral device to said energy source through said controller.

11. The patient care system according to claim 10, wherein said energy source comprises an onboard rechargeable battery, said controller recharging said battery when said controller is coupled to an external energy source, and when said controller is decoupled from said external energy source said controller supplying electrical current to said electrical outlet from said battery.

12. The patient care system according to claim 1, further comprising an energy source for powering one or more devices at or in said apparatus.

13. A patient care system comprising:
a support having an upper portion and a lower portion, said upper portion being removably mounted to said lower portion wherein said upper portion may be removed from said lower portion, said upper portion being adapted to mount to a fixture when removed from said lower portion, and said upper portion including an input connector for coupling to a monitoring device;
said lower portion of said support being adapted for being supported on a floor or ground surface; and
a controller in communication with said input connector, said input connector for coupling said controller with the monitoring device for receiving data from the monitoring device for one of (1) storing, (2) monitoring, and (3) analyzing the received data.

14. The patient care system according to claim 13, wherein said controller analyzes said received data.

15. The patient care system according to claim 14, wherein said controller analyzes trends in said received data.

16. The patient care system according to claim 14, wherein said controller is in communication with reference data, said controller analyzing said received data by comparing the received data to the reference data to determine a condition.

17. The patient care system according to claim 14, wherein said controller determines a patient condition based on said controller analyzing said received data.

18. The patient care system according to claim 14, wherein said controller generates a notification based on said controller analyzing said received data.

19. The patient care system according to claim 13, wherein said controller organizes the received data in a display.

20. The patient care system according to claim 13, wherein said controller organizes the received data in a report.

21. The patient care system according to claim 20, said controller transmitting, displaying, or making accessible said report.

22. The patient care system according to claim 20, further comprising a printer, said controller in communication with said printer and transmitting said report to said printer, and said printer printing said report.

23. The patient care system according to claim 22, wherein said printer is mounted at said upper portion of said support.

24. The patient care system according to claim 13, said controller configured for determining a suggested action for a condition based on said received or stored data.

25. The patient care system according to claim 24, said controller communicating said suggested action.

26. The patient care system according to claim 24, said controller generating actuating signals for controlling a peripheral device to implement said suggested action.

27. The patient care system according to claim 26, further comprising a display, said controller in communication with said display, and said display displaying said suggested action.

28. The patient control apparatus according to claim 13, wherein said upper portion forms a housing.
